(19) United States
(12) Patent Application Publication
(10) Pub. No.: US 2007/0232871 A1
(43) Pub. Date: Oct. 4, 2007

(54) Method and system for determining tissue properties

(76) Inventors: Edward Sinofsky, Dennis, MA (US); Jon T. McIntyre, Newton, MA (US)

Correspondence Address: FAY KAPLUN & MARCIN, LLP, 15O BROADWAY, SUITE 702, NEW YORK, NY, 10038, US

(21) Appl. No.: 11/397,123

(22) Filed: Apr. 3, 2006

(51) Int. Cl. A61B 18/18; A61B 18/04; A61B 5/00

(52) U.S. Cl. 600/310; 606/11; 606/20; 606/34

(57) ABSTRACT

A probe for detecting changes in tissue properties comprising an illumination element providing light to a target area and a sensing element receiving light from the illumination element after reflection from a target portion of tissue in combination with a device that detects changes in a property of the light received by the sensing element and determining, based on the detected changes in the property of the received light, a change in the target tissue.

### BACKGROUND

Heat is often used to treat tissue, e.g., connective tissues, tumors, fibroids, etc. In such procedures, thermal energy is delivered to a target tissue mass to, for example, shrink or necrose the tissue.

However, many current systems provide little to no feedback on the progress of the thermal treatment. Those systems which do monitor the progress of such treatments are often unable to account for parameters which affect the degree of treatment of tissue and ultrasound imaging systems which are used to monitor necrosis are not universally effective or consistent with all thermal energy sources. In addition, such systems often require specific expertise and/or elaborate equipment. Thus, it is difficult for physicians to accurately determine when a desired degree of treatment of a target tissue mass has been achieved.

### SUMMARY OF THE INVENTION

The present invention is directed to a probe for detecting changes in tissue properties comprising an illumination element providing light to a target area and a sensing element receiving light from the illumination element after reflection from a target portion of tissue in combination with a device that detects changes in a property of the light received by the sensing element and determining, based on the detected changes in the property of the received light, a change in the target tissue.

### BRIEF DESCRIPTION OF THE DRAWINGS

FIG. 1 shows an exemplary embodiment of a system, according to the present invention, comprising a spectral reflectance probe in conjunction with a tissue treatment device and a laparoscope;

FIG. 2 shows an experimental bench test set-up for determining feasibility of detecting sub-surface tissue changes using spectral reflectance;

FIG. 3 shows an ultrasound probe feasibility working model comprising illumination and sensing fibers;

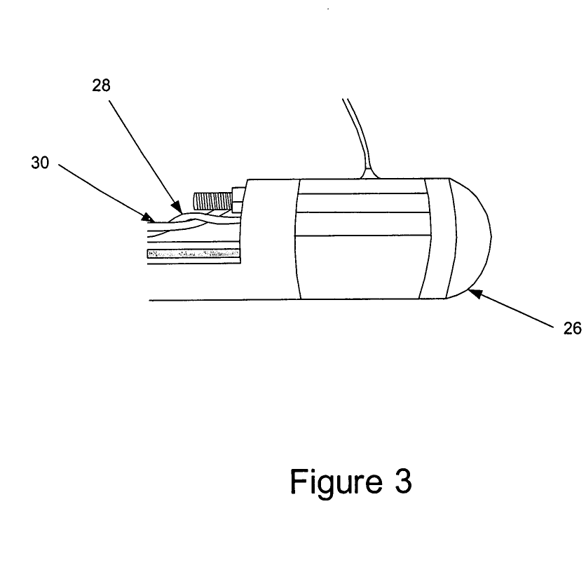

FIG. 4 shows a schematic of a system according to an alternative embodiment of the present invention.

### DETAILED DESCRIPTION

The present invention may be further understood with reference to the following description and the appended drawings, wherein like elements are referred to with the same reference numerals. The invention relates to a system using a feedback device in conjunction with a device for thermal treatment of tissue. More specifically, the invention relates to a system using a spectral reflectance probe that detects sub-surface tissue changes to determine an extent of the tissue treatment.

The system according to an embodiment of the present invention comprises a first elongated member with a treatment device and a second elongated member with a spectral reflectance probe. The two elongated members may be connectable to each other, or the two members may be completely independent. If coupled, the two elongated preferably members remain slidable relative to one another. The treatment device delivers energy to the tissue mass targeted for treatment. The spectral reflectance probe includes an illumination fiber and a sensing fiber. The illumination fiber delivers white light, or one or more specific wavelengths of light, from the distal tip of the probe, and the sensing fiber detects the light reflected from the tissue. In addition, the system may comprise a third elongated member with a laparoscope or other vision device to observe the procedure.

In preparation for tissue treatment, a trocar is inserted to the tissue treatment location and the treatment device and spectral reflectance probe are inserted through the trocar to the target tissue mass. Alternatively, the treatment device and spectral reflectance probe may be inserted to the target tissue mass through separate trocars. The tip of the treatment device is positioned at a desired location within the target tissue mass and the tip of the spectral reflectance probe is preferably positioned outside the target tissue mass so that the illumination fiber delivers light to an outer surface of the target tissue mass with the sensing fiber detecting light reflected from the tissue to establish a baseline reflectance signal. In addition, a laparoscope or other vision device may be inserted through an additional trocar to observe the procedure. Alternatively, the treatment device and spectral reflectance probe may be inserted to the target tissue directly through the skin without the use of a trocar.

During tissue treatment, the zone of treated (coagulated) tissue grows and, as an advance edge of the treated tissue expands and approaches the surface of the target tissue mass, the qualities of the light reflected from the tissue alter. Thus, these changes may be monitored by analyzing the light received by the sensing fiber and the data is conveyed to a user of the system indicating the detected change in tissue properties. Reflectance changes at one or more wavelengths may be monitored during the course of the treatment to determine when a desired level of treatment has been completed. Those skilled in the art will understand that spectral reflectance may be used in the same manner to detect changes in tissue resulting from other types of treatments including cryogenic and chemical ablations. A suitable method of detecting tissue changes is disclosed in U.S. Pat. No. 5,071,417 entitled Laser Fusion of Biological Materials, the entire disclosure of which is hereby incorporated by reference.

The ability of the spectral reflectance probe to detect tissue changes below the surface of the target tissue mass depends upon the light penetrability of the tissue mass and the depth of the tissue below the surface of the tissue mass. The illumination fiber preferably delivers a wavelength of light selected based on the tissue properties with. Wavelengths of light with deeper tissue penetrations such as, for example, 600 to 900 nm, or more preferably, 635 to 780 nm, are preferred with wavelengths such as 635, 730 and 780 nm which are commercially available being more preferable as water absorption would be reduced. As would be understood by those skilled in the art, wavelengths which penetrate more shallowly (e.g., to a depth of less than 1 cm)—i.e., wavelengths above 905 or 940 nm—may unesirably heat and damage tissue.

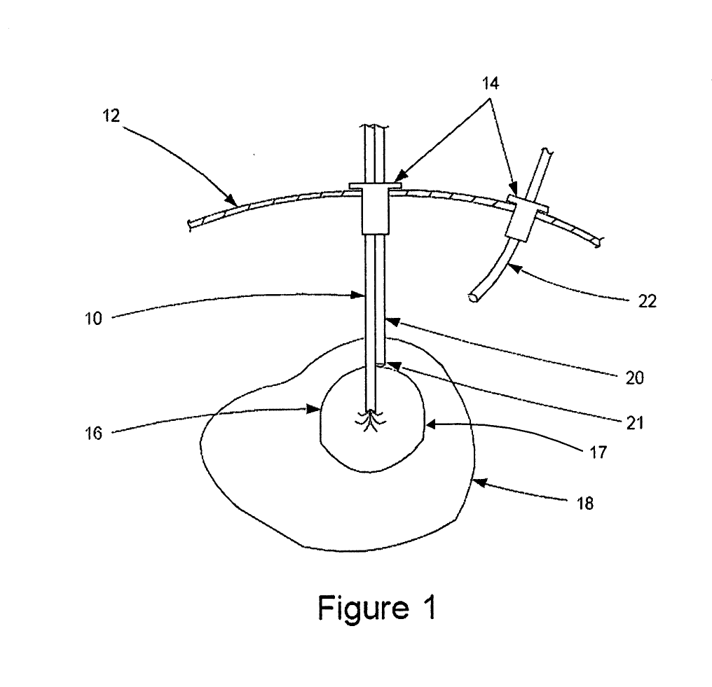

FIG. 1 shows an exemplary embodiment of a system, according to the present invention, comprising a spectral reflectance probe 20 in conjunction with a tissue treatment device 10 and a laparoscope 22. The tissue treatment device 10 which, in this embodiment is an interstitial probe including an electrode for delivering RF energy to tissue, is inserted through the skin 12 via a trocar 14. Those skilled in the art will understand that the system according to the invention will work equally well with other tissue treatment devices including ultrasound, laser, microwave, cryogenic and chemical ablation systems, etc. The tip of the treatment device 10 is inserted within a target tissue mass 16 (e.g., near a center thereof) within an organ 18 and the spectral reflectance probe 20 is inserted alongside the treatment device 10 until a distal tip 21 of the probe 20 is positioned adjacent to an external surface of the target tissue mass 16. Further, to observe the procedure and to facilitate the insertion of the treatment device 10 and spectral reflectance probe 20 to their desired locations, a laparoscope 22 may be inserted through the skin 12 using an additional trocar 14 as would be understood by those skilled in the art. Those skilled in the art will understand that certain types of treatment devices (e.g., certain ultrasound heating devices) do not need to be inserted into the center of a target tissue mass. For example, a device may focus ultrasound energy from a plurality of ultrasound crystals on a spot separated from the device to heat tissue at a distance. Those skilled in the art will understand that, depending on the distance from the device to the focus area and the size of the target tissue mass 16, such a device may be positioned adjacent the surface 17, within the tissue mass 16 but away from the center or outside the target tissue mass 16 separated from the surface 17. Such a device is described in a U.S. Patent Application entitled, “Apparatus and Method for Stiffening Tissue” filed Mar. 29, 2005 naming Isaac Ostrovsky, Michael Madden, Jon T. McIntyre and Jozef Slanda as inventors, the entire disclosure of which is hereby expressly incorporated by reference herein.

Before the treatment is begun, an illumination element 23 of the spectral reflectance probe 22 is actuated to illuminate the external surface 17 of the target tissue mass 16 and a sensing element 25 receives light reflected from the external surface 17 and transmits the light to a sensor such as a spectrometer or silicon photodetector which converts the light to an electric signal representative thereof. This electric signal is then transmitted to a controller 36 which analyzes the signal to establish a base line reflectance level for the target tissue mass 16. Once this value has been established, treatment is begun by energizing the treatment device 10 to deliver thermal energy to the center of the target tissue mass 16. As the thermal energy gradually treats the tissue mass 16 a treated portion of the tissue mass 16 expands and a leading edge of this treated portion of tissue approaches the surface 17 of the tissue mass 16. As this leading edge moves toward the surface 17, the illumination element 23 constantly or intermittently illuminates the surface 17 and the controller 36 analyzes reflectance changes of the light received by the sensing element 25 to determine the position of the leading edge relative to the surface 17. Feedback is provided to a user of the system to indicate the progress of the treatment. That is, changes in the properties of specific wavelength bands of the reflected light will indicate a degree of necrosis. For example, a spectrometer or other sensor may be used to identify the intensities of various frequency ranges of light to generate a ratio of these intensities to intensities measured before treatment was initiated to determine a rate and/or amount of change corresponding to the coagulation or necrosis of the target tissue.

Additionally, FIG. 1 shows the distal tip 21 of the spectral reflectance probe 20 positioned adjacent to the surface 17 of the target tissue mass 16. Although this configuration may be preferable for certain applications such as uterine fibroids, for other applications such as cancerous tumors, the tip 21 of the probe 20 is preferably separated from the surface 17 of the target tissue mass 16 by a short distance (e.g., 1 to 2 cm) to allow treatment and spectral reflectance monitoring to continue through the outer surface 17 to encompass a desired margin of healthy tissue surrounding the target tissue mass 16.

In cases where a previous assessment of the size of a target tissue mass 16 has been made, the use of a spectral reflectance probe 20 according to the present invention does not add any significant steps to the procedure. For example, where symptoms indicative of uterine fibroids are present, a diagnostic ultrasound is generally performed to confirm the presence of the fibroids and to determine their location and size. When the fibroids are to be treated, the treatment device 10 and a spectral reflectance probe 20 are inserted into the body side by side and the treatment device 10 is further advanced to center of the fibroid while the spectral reflectance probe 20 is positioned adjacent to an outer surface of the fibroid with the illumination element 23 and the sensing element 25 thereof facing the fibroid.

According to an embodiment of the invention, the spectral reflectance probe 20 and the treatment device 10 are slidably coupled to one another to form a single device for treating tissue and monitoring the treatment. Further, the spectral reflectance probe 20 may be incorporated as part of a disposable tissue treatment device 10.

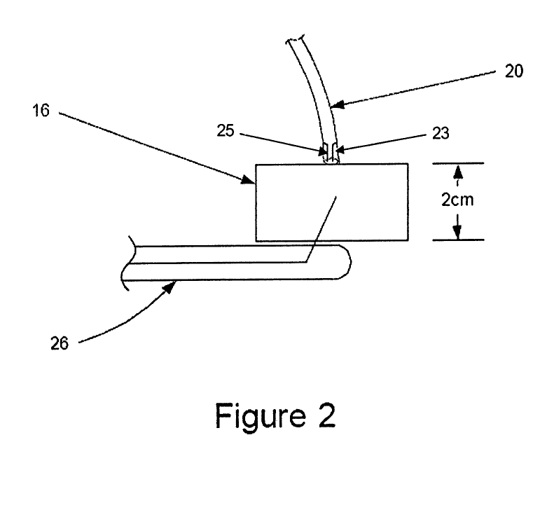

As shown in FIG. 2, target tissue 16 is located between an ultrasound probe 26 according to a further embodiment of the invention and a spectral reflectance probe 20. As described above, this probe 26 may be located within the target tissue 16 or at any point outside the tissue 16 which will allow the probe 26 to treat the target tissue 16. As with the above described embodiments, the probe 26 may be movably coupled to the spectral reflectance probe 20 in any desired manner and, depending on the qualities of the probes 20 and 26, may be rigidly coupled to one another so that a distance separating the probe 20 from the surface 17 is fixed relative to the location of the probe 26. According to this embodiment, the illumination element 23 of the spectral reflectance probe 20 includes a 20 milliwatt laser producing light of 635 nm wavelength. However, as would be understood by those skilled in the art, other wavelengths may be used to achieve a desired depth of tissue penetration although wavelengths below a range of 940 nm are preferable to minimize water absorption with wavelengths below 905 nm being more preferable. Below these values there are many commercially available wavelengths that may be used. In addition, the illumination element 23 includes an illumination fiber 28 which, in this embodiment is a 400 micron optic fiber while the sensing element 25 includes a sensing fiber 30 which in this embodiment is a 600 micron optical fiber. A spectral reflectance probe 20 constructed as described herein detects tissue changes at depths ranging from 0 to approximately 20 mm. Furthermore, the probing wavelength may be changed to enhance results for different tissue depths and may be altered during the procedure to adjust for the changing depth of the leading edge of the treated tissue. Those skilled in the art will understand that the components of the illumination element 23 and the sensing element 25 may be varied to suit the design requirements of the probe 20 and its intended use, etc. Furthermore, the details of the construction of the sensing probe 20 in regard to any of the disclosed embodiments may be rearranged in any manner as the probe 20 will operate in the same manner regardless of the type of treatment device with which it is used.

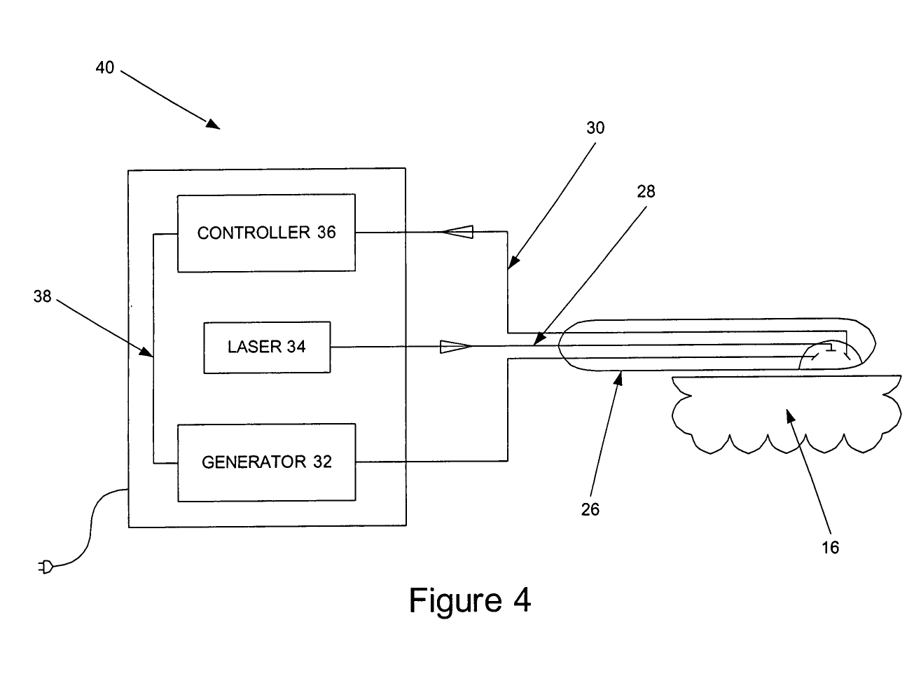

FIG. 4 shows a schematic of a non-invasive system 40 according to the present invention, comprising a generator 32, a light source 34 (e.g., a laser), and a controller 36 coupled to an integrated ultrasound probe 26 as described above comprising an illumination fiber 28 and a sensing fiber 30. Those skilled in the art will understand that the system 40 ablates tissue provides real time feedback on the degree of ablation without penetrating the target tissue mass 16. The generator 32 delivers energy to the ultrasound probe 26 for treatment of the target tissue mass 16. The light source 34 provides illumination of the target tissue mass 16 via the illumination fiber 28 and the controller 36 receives and analyzes spectral reflectance data transmitted thereto from the probe 20 via the sensing fiber 30. Thus, the illumination and sensing fibers 28, 30, respectively, for detecting spectral reflectance are integrated into a single ultrasound probe 26 for simultaneous tissue treatment and detection of spectral reflectance.

As would be understood by those skilled in the art, the generator 32 delivers energy to the ultrasound probe 26 to stimulate vibration of one or more crystals (not shown) of the ultrasound probe 26 to treat a target tissue mass 16. Simultaneously, the light source 34 delivers light to the surface of the target tissue mass 16 through the illumination fiber 28, either continuously or at desired intervals, while the controller 36 receives reflectance changes of the target tissue mass 16 through the sensing fiber 30. The controller 36 may optionally analyze reflectance changes of the tissue mass 16 and control, via the feedback loop 38, energy delivery by the generator 32. Thus, the system 40 may regulate and ultimately terminate tissue treatment based on reflectance changes of the target tissue mass 16 automatically reducing or eliminating the potential for user errors and reducing the actions required of the user.

The exemplary embodiment described above in conjunction with FIG. 1, uses radio frequency energy as the tissue treatment thermal energy source. However, the system of the present invention using spectral reflectance may be used with many other tissue treatment thermal energy sources, including but not limited to microwave and laser energy. In addition, the exemplary embodiments described above have discussed treatment of cancerous tumors and uterine fibroids. Other potential applications for the spectral reflectance probe of the present invention include, but are not limited to, prostate cancer, benign prostatic hypertrophy (BPH).

The embodiment described in regard to FIG. 4 is particularly suited for the treatment of stress urinary incontinence via transvaginal delivery of ultrasound energy to create subsurface tissue effects without penetrating the surface. Other potential applications for this embodiment include, among others, gastroesophageal reflux disease (GERD), fecal incontinence, joint conditions such as rotator cuff injuries, and cosmetic applications such as treating wrinkles.

The present invention has been described with reference to specific embodiments, and more specifically, with reference to a system comprising a spectral reflectance probe for use during tissue treatment. However, other embodiments may be devised that are applicable to other devices and procedures, without departing from the scope of the invention. For example, the sensing element may include any electronic imaging device sending electrical signals directly to the controller. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A probe for detecting changes in tissue properties comprising:
an illumination element delivering light to a target area;
a sensing element receiving light after reflection from a target portion of tissue; and
a controller detecting changes in a property of the light received by the sensing element and determining a change in the target tissue.

2. The probe according to claim 1, wherein the illumination element includes an optic fiber coupled to a laser.

3. The probe according to claim 2, wherein light delivered by the laser has a wavelength of less than approximately 940 nm.

4. The probe according to claim 3, wherein the light delivered by the laser has a wavelength less than approximately 905 nm.

5. The probe according to claim 3, wherein the light delivered by the laser has a wavelength of approximately 730 nm.

6. The probe according to claim 3, wherein the light delivered by the laser has a wavelength of approximately 635 nm to 670 nm.

7. The probe according to claim 1, wherein the sensing element includes an optic fiber coupled to a sensor which generates an electric signal corresponding one or more properties of the light, the sensor being coupled to the controller.

8. The probe according to claim 3, wherein the light delivered by the illumination element is full spectrum white light.

9. A system for treating tissue, comprising:
a tissue treatment device altering a property of a target portion of tissue;
a probe for detecting changes in the tissue property, the probe including an illumination element focusing light on one of the target tissue and tissue adjacent to the target tissue and a sensing element receiving light from the illumination element after reflection from the target tissue and a detector detecting changes in a property of the light received by the sensing element and determining, based on the detected changes in the property of the received light, a change in the tissue property.

10. The system according to claim 9, wherein the tissue treatment device includes an ablation element.

11. The system according to claim 10, further comprising a source of RF energy wherein the ablation element includes an electrode.

12. The system according to claim 10, wherein the ablation element includes a cryogenic device.

13. The system according to claim 10, wherein the ablation element includes an ultrasound element.

14. The system according to claim 10, wherein the ablation element includes a source of microwave energy.

15. The system according to claim 10, wherein the ablation element includes a laser.

16. The system according to claim 13, wherein the ultrasound element includes an array of ultrasound elements arranged to focus ultrasound energy at an area separated from the ultrasound element by a predetermined distance.

17. The system according to claim 10, wherein the tissue treatment device ablates tissue and the tissue property is a depth of a leading edge of a region of necrosed tissue.

18. The system according to claim 10, wherein the illumination element includes an optic fiber coupled to a laser.

19. The system according to claim 18, wherein light delivered by the laser has a wavelength of less than approximately 940 nm.

20. The system according to claim 19, wherein the light delivered by the laser has a wavelength less than approximately 905 nm.

21. The system according to claim 19, wherein the light delivered by the laser has a wavelength of approximately 635 nm.

22. The system according to claim 10, wherein the sensing element includes an optic fiber coupled to a sensor which generates an electric signal corresponding one or more properties of the light, the sensor being coupled to the detector.

23. The system according to claim 9, wherein the illumination element delivers full spectrum white light.

24. The system according to claim 9, wherein the illumination element includes a laser delivering light with a wavelength of approximately 730 nm.

25. The system according to claim 9, wherein the illumination element includes a laser delivering light with a wavelength of approximately 780 nm.

26. The system according to claim 9, wherein the illumination element includes a laser delivering light with a wavelength of approximately 670 nm.

27. A method of treating tissue comprising:
ablating tissue within a target tissue mass; and
illuminating the target tissue mass;
detecting light reflected from the target tissue mass; and
analyzing the detected light to determine a depth of ablated tissue within the target tissue mass.
